(19) United States
(12) Patent Application Publication
(10) Pub. No.: US 2023/0045688 A1
(43) Pub. Date: Feb. 9, 2023

(54) CUSTOMMUNE: A WEB TOOL FOR DESIGNING PERSONALIZED AND POPULATION-TARGETED PEPTIDE VACCINES

(71) Applicants: Tarek Mohammad, Cairo (EG); Iart Luca Shytaz, Sao Paulo (BR); Mahmoud Elhefnawi, Cairo (EG); Andrea Savarino, Vinovo (IT); Ricardo Sobhie Diaz, Barueri (BR)

(72) Inventors: Tarek Mohammad, Cairo (EG); Iart Luca Shytaz, Sao Paulo (BR); Mahmoud Elhefnawi, Cairo (EG); Andrea Savarino, Vinovo (IT); Ricardo Sobhie Diaz, Barueri (BR)

(21) Appl. No.: 17/901,094

(22) Filed: Sep. 1, 2022

Related U.S. Application Data
(63) Continuation of application No. PCT/IB2021/051732, filed on Mar. 2, 2021.
(60) Provisional application No. 62/984,318, filed on Mar. 3, 2020.

(51) Int. Cl. A61K 39/215; A61K 39/00; A61K 39/21; A61K 39/39; G16H 70/40

(52) U.S. Cl. CPC A61K 39/215; A61K 39/0011; A61K 39/21; A61K 39/39; G16H 70/40; A61K 2039/5258; A61K 2039/55505; A61K 2039/60; A61K 2039/70

(57) ABSTRACT

Computational prediction of immunogenic epitopes is a promising platform for designing therapeutic and preventive vaccines. A potential target is, for example, the human immunodeficiency virus (HIV-1) for which, despite decades of efforts, no vaccine is available. Indeed, due to the enormous variability of the virus, a single formulation effective against all or most HIV strains might not be achievable. Moreover, upon infecting host cells, HIV-1 can integrate in the host genome and form long lasting latent reservoirs that are not susceptible to common antiretroviral treatments. Therefore, a therapeutic vaccine designed to eliminate infected cells might represent a key component of strategies aimed at curing the infection. We herein introduce an automated algorithm to produce personalized and population-based vaccines.

### BACKGROUND OF THE INVENTION

Computational prediction of immunogenic epitopes is a promising platform for designing therapeutic and preventive vaccines. A potential target is, for example, the human immunodeficiency virus (HIV-1) for which, despite decades of efforts, no vaccine is available (Burton 2019; Stephenson 2018). Indeed, due to the enormous variability of the virus, a single formulation effective against all or most HIV strains might not be achievable. Moreover, upon infecting host cells, HIV-1 can integrate in the host genome and form long lasting latent reservoirs that are not susceptible to common antiretroviral treatments (Churchill et al. 2016). Therefore, a therapeutic vaccine designed to eliminate infected cells might represent a key component of strategies aimed at curing the infection. Peptides designed on the basis of individual viro-immunological features of HIV⁺ individuals have recently shown the ability to induce post-therapy viral set point abatement (Diaz et al. 2019). However, the reproducibility and scalability of this method has been curtailed by the need to manually intersect virologic and immunologic data for each patient and by potential arbitrariness in selecting between different peptide vaccine candidates.

We herein introduce an automated algorithm to produce personalized and population-based vaccines, applicable not only against HIV but also against other RNA viruses and various types of cancer.

### SUMMARY OF THE INVENTION

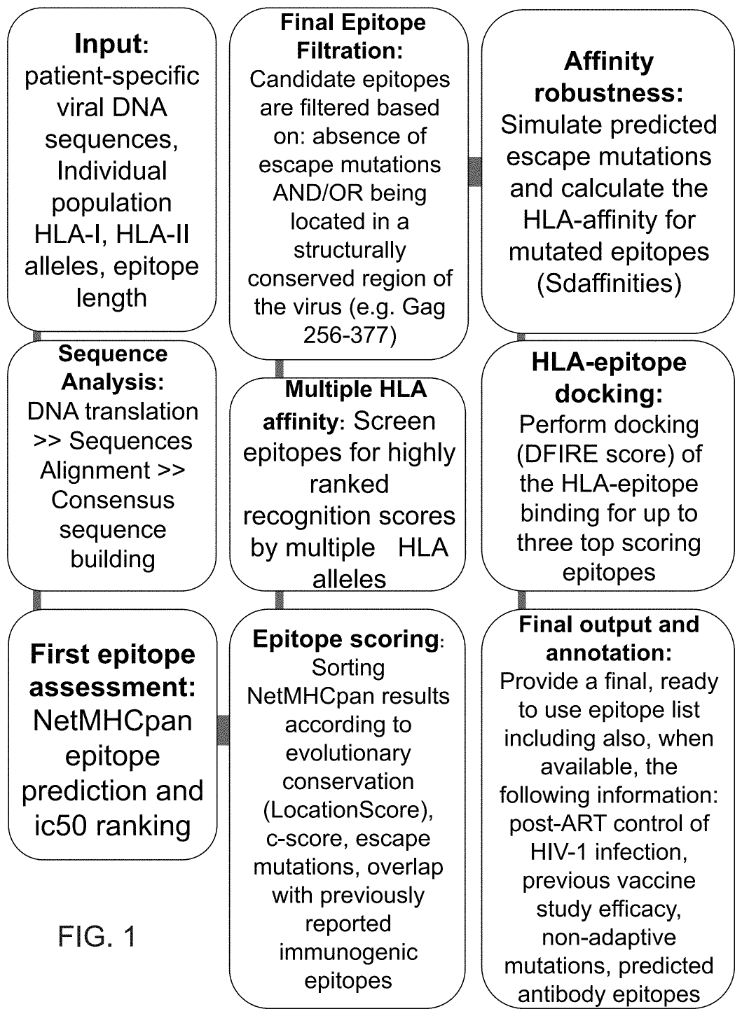

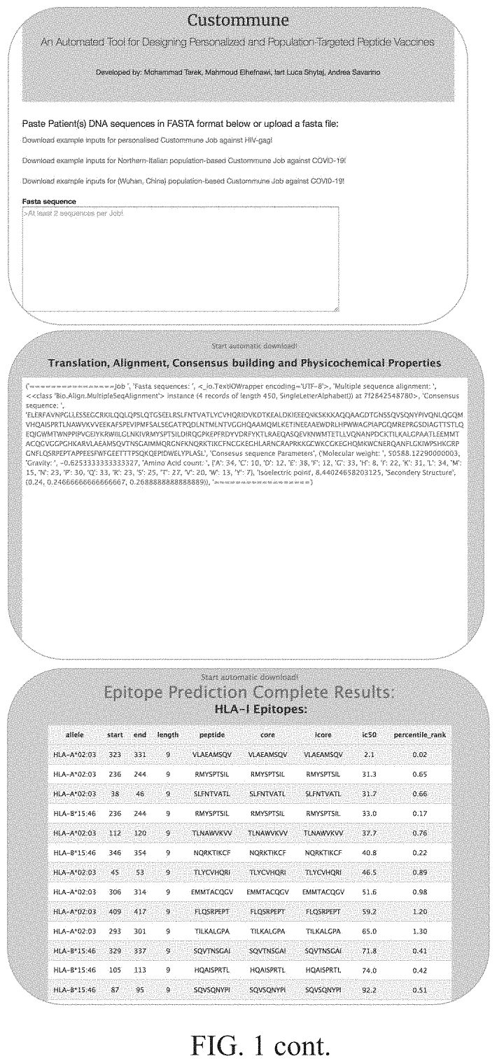

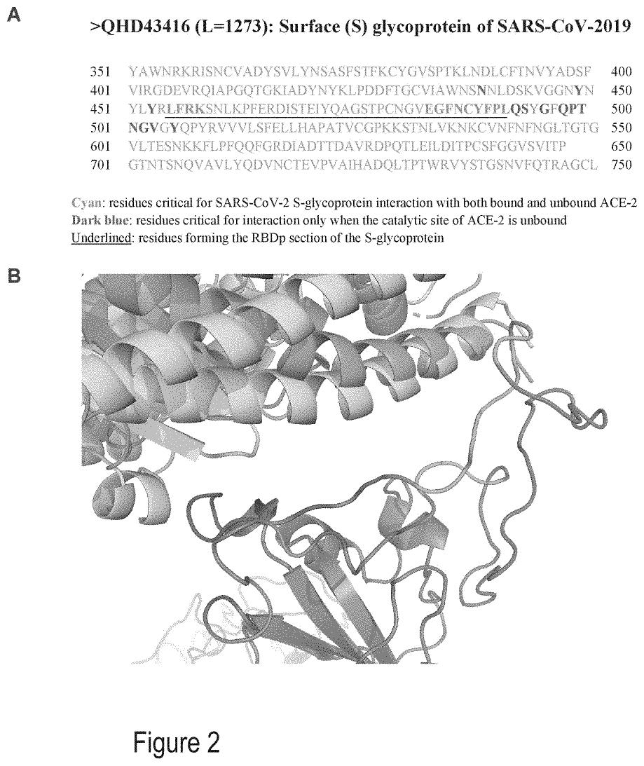

1) The present invention mainly focuses on the calculation of the scores used for ranking the peptides to be chosen for use in personalized vaccines. The algorithm proposed is completely independent of the software used and allows an accurate prediction of the immunogenic peptides that need to be chosen in order to ensure optimal binding to the candidate vaccine's HLA antigens. Previous inventions aimed at calculating the optimal peptides for presentation to lymphocytes through the vaccine's HLA antigens did not apply the same scoring system. The Custommune score that we propose is based on the affinity scoring of the final top-scoring peptides by structurally fitting them into the binding groove of their predicted HLA allele, allowing the pipeline to check the reliability of the theoretical IC50 values. The pipeline compares them to the structural docking scores which should show higher affinity and lower binding energies with low theoretical IC50 values. It then helps the final scoring function to prioritize the recommended list of epitopes in an attempt to decrease false positives. This goes in parallel with computing a standard deviation of the predicted theoretical IC50 values for theoretical mutants of top peptides, allowing the tool to consider peptides with less potential to lose affinity due to possible mutations in their sequences (FIG. 1). This is of particular importance when target proteins have high mutation frequencies such as in the case of RNA viruses and cancer (FIGS. 2-5).

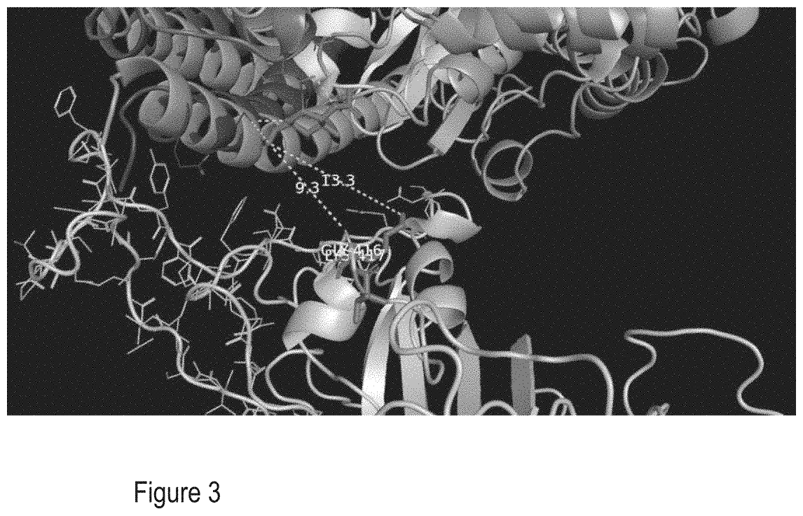

2) The vaccine design pipeline proposed in the present invention allows population-targeted approaches. Depending on the HLA allele, frequencies in publicly available databases and population-specific studies, Custommune receives a set of alleles selected on the basis of weighted frequencies of highly frequent alleles (>0.1% of each population dataset). These frequencies are also used to estimate a theoretical population coverage for the final construct. With this approach a vaccine is proposed, based on sequences of the SARS-CoV-2 surface glycoprotein responsible for its binding with the main receptor of the virus on the cell surface (FIGS. 2 and 3).

3) Finally, the present invention provides an automated system for T-cell epitope prediction (FIG. 1), resulting in potential vaccines, instead of following a time consuming process involving multiple steps and a certain degree of expertise in bioinformatics. This automated pipeline is a user friendly interface that can be adopted by any operator having only basic knowledge in biology. This application could represent an advantage in the future when personalized vaccines will be widely adopted in many medical settings in the world, which, given the amount of workload, will require operators with only an intermediate level of expertise, such as nurses or medical doctors with no specific training in molecular medicine.

### DETAILED DESCRIPTION OF THE FIGURES

FIG. 1. Illustrated workflow of Custommune epitope prediction

(Input) the Custommune pipeline starts by validating user inputs for sequences, alleles and desired epitope length. (Sequence analysis) input sequences are then translated to build an alignment of amino acid sequences from which a consensus sequence is generated and used for further epitope prediction. (First epitope assessment) using the netMHCpan 4.0 algorithm 35, Custommune initially ranks epitope predictions based on their IC50 values. (Epitope scoring) additional scoring layers are then applied by Custommune based on: location of the epitope (by assigning a LocationScore to epitopes located in an evolutionary conserved region); evolutionary conservation of the epitope residues (C-Score) assessed by using an internal sequence database (Supplementary File 1) or the Basic Local Alignment Search Tool (BLAST; https://blast.ncbi.nlm.nih.gov/Blast.cgi); presence of reported escape mutations; overlap with previously reported immunogenic epitopes (DOverlap) retrieved using an internal database. (Multiple HLA affinity) following these filtration layers, Custommune identifies whether any predicted epitope displays high-affinity to multiple HLA alleles and (Final epitope filtration) discards any epitopes that have reported escape mutations and/or are not located in an evolutionary conserved region. (Affinity robustness) among remaining candidates, Custommune restricts further analyses on the three top scoring epitopes for both HLA classes. For these, Custommune computes the HLA binding affinities of potential mutant versions, though not classified as escape mutations, to estimate the impact of these mutations on epitope recognition (SDaffinities). (HLA-epitope docking) on the same three top ranking epitopes, Custommune computes epitope-HLA allele docking scores, calculated using the LightDock79 python package and scored using the DFIRE85 scoring function. (Final output and annotation) in a parallel process, the Bepipred 2.039 algorithm is implemented to predict neutralizing antibody epitopes from the initial consensus sequence, that can be further intersected with Class II restricted epitopes to increase immunogenicity. As a final output, for both Class I and II HLAs, Custommune ranks the top 3 epitopes according to a score (CustoScore) which accounts for all aforementioned filtration parameters.

FIG. 2. Identification of vaccine targets in the receptor binding domain (RBD) of the SARS-CoV-2 Spike (S) glycoprotein.

(A) Partial sequence of the SARS-CoV-2 S-glycoprotein (derived from structure QHD4341690). Residues constituting the protein-protein interaction surface of the S-glycoprotein (magenta) with ACE2 are shown in different gradations of blue. Residues responsible for binding of the S-glycoprotein only in the presence of an unbound catalytic site of ACE2 are shown in dark blue. The residues underlined correspond to the receptor binding domain 1 (RBDp), as described in the main text. (B) Interaction of SARS-CoV-2 S-glycoprotein (magenta) with superimposed structures of unbound ACE2 (yellow) or ACE-2 bound to the competitive inhibitor MLN-4760 (green). The specific segment in the receptor binding domain (RBD) of the S-glycoprotein that was found to overlap with both configurations of ACE2, i.e. unbound catalytic domain or catalytic domain bound with inhibitor MLN-4760, is shown in cyan. Residues binding only to unbound ACE-2 are shown in dark blue.

FIG. 3. Spike glycoprotein of SARS-CoV-2 (cyan) interacts with superimposed structures of ACE2 in both states: bound with the competitive inhibitor MLN-4760 (blue) and unbound, inactive, state (green). N-Acetyl-D-glucosamine (NAG) shown in red was found to be in close proximity to the interaction interface between the spike glycoprotein and ACE2. NAG was found to bind Lys26 and Asn90 on ACE2 and Gly416 and Lys417 on the spike glycoprotein

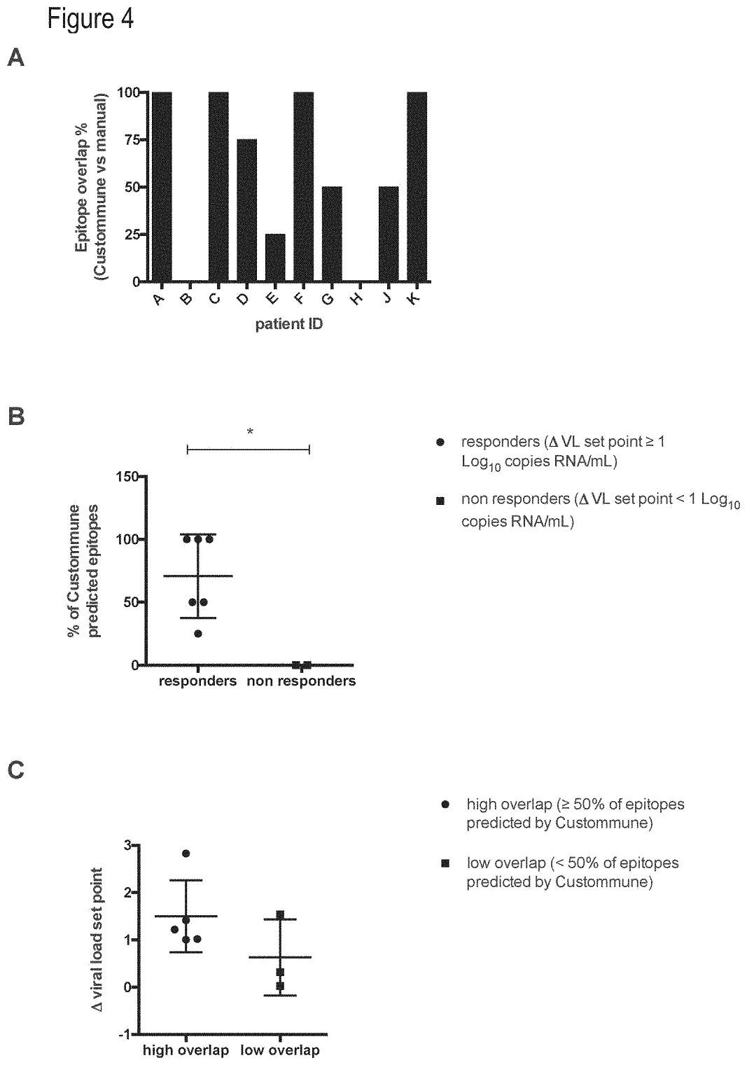

FIG. 4. Potential therapeutic efficacy of Custommune-predicted vaccine candidates.

(A) Percentage of personalized peptides predicted by Custommune which overlap with those administered as vaccines to people living with HIV/AIDS (PLWHA) in clinical trial NCT02961829. Each letter indicates a trial participant. (B) Percentage of overlap between epitopes predicted by Custommune and epitopes administered in the trial in virologic responders and non responders. Virologic responders were defined as individuals with Δ viral load set point ≥1 Log 10 copies of HIV-1 RNA/mL of plasma. Data were analyzed by two-tailed Student t-test. Panel C) Δ viral load set point in trial participants who received peptides with high or low overlap to Custommune predictions (≥50% or <50% overlap, respectively).

The Δ viral load set point was calculated as the difference between pre- and post-therapy viral load set points, with post-therapy viral load set point calculated as the median of all available measurements (up to 9 weeks post-treatment interruption). Each data point in panels B and C indicates a trial participant.

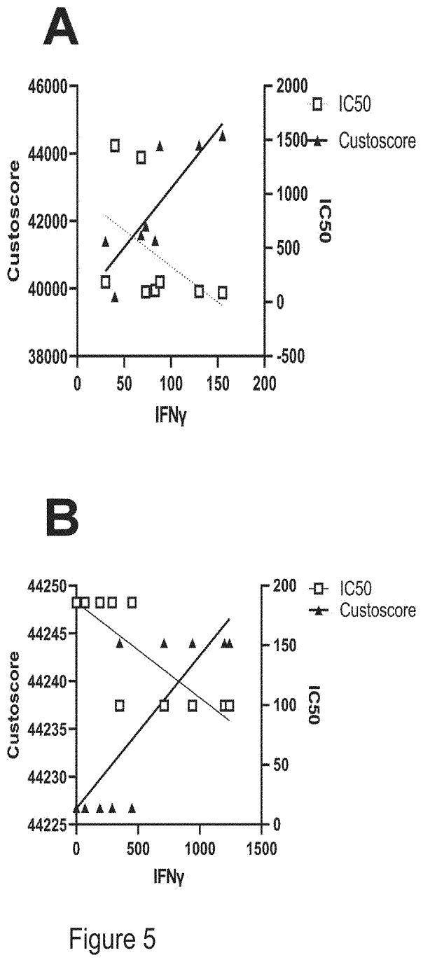

FIG. 5. Correlation between in-vivo IFNγ CTL responses from immunized HLA-B*27 mice and Custommune predictions for selected pool of MUC1 peptides. Splenocytes from 2 mice were restimulated with 2 MUC1 peptides (A), and CD4-depleted splenocytes from five mice were restimulated with four different peptides (13).

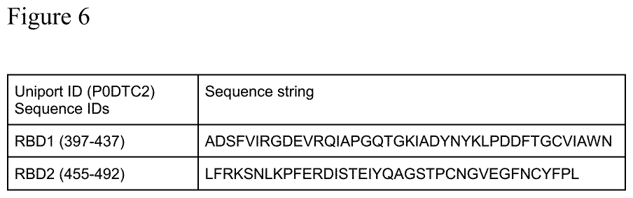

FIG. 6. Sequence string for RBD.

### DETAILED DESCRIPTION OF THE INVENTION

Custommune is a user-friendly web tool that streamlines a thorough pipeline (FIG. 1) for designing epitopes for preventive and therapeutic vaccines.

Written in Python (Python Software Foundation, version 3.7. Available at http://www.python.org) using the Django framework (version 2.2.6 https://www.djangoproject.com/start/overview/), Custommune provides the user with an easy online interface for accessing and downloading prediction datasets without any coding knowledge requirements.

For HIV-1 vaccine design, the tool intersects input data from patient-specific viral sequences (DNA in FASTA format or raw DNA sequencing inputs) and patient's HLA-I and/or HLA-II alleles, giving an output of epitopes of desired k-mer length. Even though the approach could be potentially extended to encompass entire HIV-1 sequences, so far only the gag gene has been used to infer viral epitopes with Custommune. This is motivated by the unique features of anti-gag cell mediated immune responses, which were repeatedly highlighted as a correlate of viral load decrease in HIV⁺ individuals and of post-therapy control in macaques (Kiepiela et al. 2007; Shytaj et al. 2015; Riviére et al. 1995; Zuniga et al. 2006; Jia et al. 2012).

The HLA-specific epitopes provided by Custommune are filtered according to a set of parameters that compute epitope affinity in terms of sequence variations and conservation degree, allele-restricted affinities and previous clinical evidence of immune response. The tool pipeline (FIG. 1 and Example 1) also computes related physicochemical parameters of the personalized epitope sequence to aid assessment of the structural stability of candidate peptides. Overall, the tool is optimized to identify immunogenic gag peptides characterized by the lowest variability (mutation potential). In line with this, the tool specifically highlights potential epitopes that are contained in regions which are essential for viral fitness. These regions were previously identified through in-silico and ex-vivo studies, showing that showed gag segments which are essential for viral packaging and assembly are structurally and evolutionary conserved, displaying low Shannon entropy both in humans and primate lentiviruses (Shytaj and Savarino 2015).

In another embodiment, Custommune may be used to develop novel anticancer treatments using a similar approach to that described for HIV-1. Cancer neo-epitopes are promising targets for immunotherapy (Bethune and Joglekar 2017). These peptides include specific somatic mutations that could be targeted using cellular immunotherapies or vaccine formulations to render tumors more accessible by the immune system.

Custommune could accelerate detection of cancer neo-epitopes in a personalized fashion. To this aim, the input for the tool could be derived from a library of neoantigen sequences which would be specific for the type of cancer considered. Datasets available in the literature could be used to build this library, including signature mutations, pathway analysis, frequently mutated genes and differential expression of the putative antigens. This would allow Custommune to rank a set of neoantigens specific for the phenotype of interest and match them with patient-specific HLA alleles.

While chronic or long-term conditions such as HIV-1 and cancer are ideal models for personalized vaccines, an acute life-threatening infection is more suited for population-based vaccine design. Custommune predictions could identify epitopes which are expected to be recognized and bound by the HLA haplotypes most prevalent within a population or subpopulation. This could allow sparing time and resources for sequencing and peptide production targeting single individuals and centralize standardized vaccine production at a national level. While complete herd immunity would be far from assured, having a certain proportion of the susceptible population protected might be sufficient to limit the spread of the epidemics.

An ideal model for the design of population-targeted peptides for vaccination is the severe acute respiratory syndrome coronavirus 2 (SARS-CoV-2), the pathogen responsible for the recent, and currently ongoing, CoVid19 epidemic (Velavan and Meyer 2020). SARS-CoV-2 represents an urgent challenge for vaccine development (Zhang and Liu 2020). The virus was initially reported in the Hubei province (China) in November 2019 and, since then, has caused a growing epidemic with pandemic potential (Velavan and Meyer 2020). While some antiviral agents have been proposed for use against coronaviruses (Vincent et al. 2005) and have displayed some efficacy in pilot clinical trials against SARS-CoV-2 (Gao, Tian, and Yang 2020; Savarino et al. 2006; Wang et al. 2020), no vaccine against this virus is as yet available. Interestingly, SARS-CoV-2 shares approximately 80% sequence identity with SARS-CoV (Zhou et al. 2020), the virus that was responsible for an epidemic burst of acute pneumonia in China in 2003. However, vaccine approaches attempted so far against SARS-CoV, including the use as an immunogen of the recombinant viral spike glycoprotein, which the virus uses to dock at the target cell, have not been successful as of date. In light of its user-friendly and fast interface, the Custommune pipeline (FIG. 1) can be applied to test target sequences of SARS-CoV-2 and predict optimal immunogens for HLA-s typical for the populations of each affected region or for generation of neutralizing antibodies. The present invention is however independent of the web interface that we propose, and the single steps can be performed manually or with any other computational tool.

Input patient-specific gag sequences can be copied as raw sequences or added in FASTA format to the tool. The input form also allows the user to provide the patient's phenotypic alleles both for class-I and/or class-II in either one allele per prediction or in multiple-alleles format. In addition, the user can also input the desired lengths required for the target HLA-I alleles inserted into the tool form. To facilitate the allele input step, the tool provides two links, directing the user to a list of supported HLA alleles, mirroring those of the netMHCpan 4.0 algorithm (Jurtz et al. 2017), for each HLA class.

The tool pipeline (FIG. 1) starts by translating user-input gag sequences to protein sequences. Custommune then performs a multiple sequence alignment of these sequences using the Clustal Omega (REST) web service Python client (Sievers et al. 2011) and, eventually, builds a consensus translated sequence that will be used for epitope prediction. The consensus sequence is used to predict epitopes restricted to patient-specific HLA-alleles for both classes.

Resulting epitopes are then filtered according to predicted binding strength and evolutionary conservation. For the former, epitopes are ranked in ascending order according to their IC50 values, using a cutoff of 1000 nM. For calculating evolutionary conservation, each epitope is compared for similarity to an internal database of gag sequences (detailed in the methods) collected mainly from curated gag alignments retrieved from the Los Alamos HIV sequence database (http://www.hiv.lanl.gov/). Moreover, to verify whether the antigenicity of the candidate epitopes has already been described, the tool compares potential epitopes to those already described in the Los Alamos HIV immunology site (http://www.hiv.lanl.gov/content/immunology). The overlaps are listed in a separate text to enable the user for further manual checkup. Finally, to further refine the structural assessment of epitope binding to HLA-alleles the tool performs structural epitope modelling followed by epitope-HLA docking to determine the structural stability of the HLA-predicted epitope binding.

Custommune is also designed to answer some of the clinically relevant questions about epitope ranking, one of which is determining epitopes that could exert high binding affinities for multiple alleles. Another relevant question is whether epitopes may include any previously reported escape mutations which could render the infected cells hidden from immunity. For instance, mutant versions of one epitope that exert lower binding affinities than predicted for the original epitope could indicate a potential immune-escape effect needing exclusion from the vaccine strategy. To account for this, Custommune estimates the binding affinities of the mutant versions, if any can be predicted, for the top three epitopes. Of note, when compared with manually designed peptides for personalized therapeutic vaccines against HIV-1, Custommune predictions correlated with therapeutic efficacy (Example 2)

To apply Custommune for population-targeted vaccine design against SARS-Cov-2, it is first essential to determine the target sequences of SARS-CoV-2 that could serve as a basis for predicting epitopes for HLA or neutralizing antibody recognition. Neutralizing antibodies might be preferable for this application, since they are regarded as more effective for prevention (Zhu et al. 2007), in contrast to cell mediated immunity, which is more suitable for therapeutic vaccination.

As a starting point to identify potential input sequences for Custommune, we analyzed therapeutic strategies suggested to inhibit SaRS-CoV replication, in line with the similarity between SARS-Cov-2 and SARS-CoV (Zhu et al. 2007). In particular, two previous therapeutic approaches were considered: 1) the use of neutralizing antibodies which block the portion of the S-glycoprotein that mediate the main protein-protein interaction with the cellular entry receptor, i.e. angiotensin converting enzyme 2 (ACE2) (Zhu et al. 2007); 2) the use of 4-aminoquinoline, chloroquine, which was used for decades as an antimalarial drug and was recently showed to effectively inhibit SARS-CoV-2 in vitro (Wang et al. 2020) and to have curative potential in the infected individuals (Gao, Tian, and Yang 2020). Several mechanisms have been postulated for the anti-coronavirus effect of chloroquine, the best documented of which is inhibition of ACE2 glycosylation, decreasing S-glycoprotein binding affinity and suggesting that carbohydrate moieties also contribute to SARS-CoV attachment to target cells along with the aforementioned protein-protein interaction (Vincent et al. 2005; Savarino et al. 2006).

To translate these approaches to vaccine design we:
1) performed a thorough analysis for molecular complexes of SARS-CoV-2 spike glycoprotein with the entry receptor ACE2. Considering the configuration of ACE2, we superimposed complexes of S-glycoprotein/ACE2 in both states of ACE2, i.e. a bound state with the catalytic site engaged (by angiotensin or the competitive inhibitor MLN-4760 (Dales et al. 2002)) or the free state. The analysis of the receptor binding domain of the viral spike protein (RBD) in complex with ACE2 indicated that the binding surface of the S-glycoprotein is relatively larger with the unbound configuration of ACE2 and restricted by ACE2 catalytic site engagement (FIG. 2). Therefore, we decided to focus the epitope search within the RBD sequence interacting with the catalytic site-bound ACE2, which we named RBD1 (FIG. 6). Of note, this binding surface, though small, entirely overlaps with a portion of the S-glycoprotein attached to unbound ACE2 (FIG. 2). Therefore, it is expected that this approach will be able to evoke antibodies against the RBD irrespective of the ACE2 configuration.
   This approach constitutes a significant novelty of the present invention in comparison with previously followed approaches.
2) inspected the possible contribution of oligosaccharide moieties of ACE2 to the S-glycoprotein/ACE2 binding interface, a so far unexplored topic. It is known that oligosaccharide moieties containing sialic acid and bound to ACE2 are fundamental for optimal infectivity of SARS-CoVs (Vincent et al. 2005), because disruption of oligosaccharide formation using the broad-spectrum antiviral chloroquine significantly decreases SARS-CoV infectivity (Vincent et al. 2005; Savarino et al. 2006). Despite the lack of structural data on S-glycoprotein/ACE2-bound oligosaccharide interactions, insight into this phenomenon can be obtained by analysing a published structure of ACE2 (1R4L), which presents an N-acetylglucosamine (NAG) remaining from the oligosaccharide originally attached to the protein. The NAG interacts with the ACE2 amino acid Asn90, a residue in close proximity to Lys26, which is in turn responsible for the attachment of N-acetylglucosamine moiety to ACE2. By superimposing the S-glycoprotein with the 1R4L ACE2 structure, we were able to determine the specific segment of the S-glycoprotein RBD that could be responsible for the interaction with ACE2 by measuring the atomic distances at the binding interface between the (NAG) moiety and the S-glycoprotein. Two specific residues (Gly416-Lys417) were found to interact directly with NAG (FIG. 3). Thus, by selecting a peptide that includes 20 amino acids in both forward and backward direction of the translation frame from a starting point of Gly-416, we could select a segment of the S-glycoprotein RBD—namely (RBD2) (FIG. 6), which is another bona fide optimal target for designing neutralizing antigenic epitopes to disrupt S-ACE2 cellular entry complex.

Therefore, the RBD1 and RBD2 DNA sequences of SARS-CoV-2 can be used as optimal inputs for Custommune, along with the sequences of HLA-II alleles which have been previously associated with susceptibility to infections with coronaviruses (Hajeer et al. 2016; Yang et al. 2009; Xiong et al. 2008). Indeed, by inspecting the results of both HLA-II epitope prediction and antibody epitope prediction —using Bepipred-2.0—(Jespersen et al. 2017), Custommune was able to identify four potential neutralizing epitopes —three against RBD1 (“SNLKPFERD”, “TEIYQAGSTPCNGVEG” and “LQSYGFQP”) and one against RBD2 (“IRGDEVRQIAPGQTGKIADYNYKLPD”)—that also overlap with predicted highly ranking class-II HLA epitopes.

The predicted epitopes can be included in multitargeted vaccine approaches, such as multi-epitope proteins. These can be obtained by covalently linking the neutralizing antibody epitopes to contiguous cytotoxic T-lymphocyte (CTL) epitopes, which can also be derived using Custommune. Linkage of the different epitopes can be performed using linker peptides containing proteolytic cleavage sites (Arai et al. 2001). The different neutralizing antibody and CTL epitopes can also be simultaneously linked to different portions of self-assembling peptide cages in order to increase antigenicity (Morris et al. 2019). In a further attempt to mimic the successful inhibition of SARS-CoV-2 by chloroquine, one or more CTL epitopes may be derived from the viral papain-like protease (PL-pro), which has been recently suggested as an additional target of this drug (Arya et al. 2019). The present invention is, however, not restricted to CTL epitopes derived from PL-pro, and epitopes derived from other non-structural and structural viral antigens may be flanked to the neutralizing antibody epitopes. We used the tool to predict and filter possible neutralizing epitopes against PL-pro of SARS-CoV2 by mainly focusing on the specific catalytic domain. This domain aids assembly of viral vesicles required for SARS-CoV2 replication and antagonizes type I interferon and NF-kappa-B of host cells (Clementz et al. 2010). Based on the structural analysis of SARS-CoV-2 PL-pro reported by (Arya et al. 2019) we focused our predictions on epitopes spanning the catalytic triad residues of PL-pro including; Cys114, His275 and Asp289. Custommune predictions for neutralizing antibody epitopes in the PLpro of SARS-CoV-2 returned 4 results; KTVGELGDV, YEQFKKGVQIPCTC, GNYQCGHYKHITSKET and YCIDGALLTKSSEYKGPIT. Of these, GNYQCGHYKHITSKET encompasses the catalytic residue His275 while epitope YCIDGALLTKSSEYKGPIT encompasses Asp289.

In addition, suitable commercially available adjuvants can be used for vaccine administration. These may include, but are not restricted to, water-in-oil or oil-in-water or double layered emulsions, and suitable polymers, in particular those containing TLR ligands to increase epitope-driven immune activation (Li et al. 2014; Lei et al. 2019).

Finally, for detection of cancer neo-epitopes, a procedure similar to that detailed for HIV-1 can be applied. Specifically, the Custommune pipeline will process input reads by aligning them to reference sequences, followed by mutation detection and building of consensus peptide sequences. Subsequently, Custommune will perform in-silico epitope prediction from consensus peptides. These potential epitopes will be ranked according to allele-restricted affinities for neoepitopes and the difference between affinities of neoepitopes and corresponding non-mutated versions. Then, highly ranking epitopes will be subjected to further filtration layers considering: mutation site, peptide conservation, mutation frequency, predicted functional deleteriousness of mutations, overlapping with internal neoepitope database items and stability of the neoepitope and its structural binding to the restricted allele.

The tool will report back a scoring report for highly ranking filtered candidates reflecting a scoring function that considers neoepitope-identification parameters. The tool will also provide corresponding DNA sequences for candidate epitopes to facilitate delivery through vaccine-adjuvants and/or engineered cellular therapies.

To conclude, Custommune provides the user with an automated pipeline for personalized or population-targeted peptide vaccine design using a multilayer epitope filtration approach. The tool also provides the user with the ability to download and inspect sequence translation data, sequence alignment data and consensus sequences generated with its computed physico-chemical parameters, including secondary structure predictions to allow the user to manually assess the stability of consensus sequences. Custommune outputs can be further downloaded as ranked epitope prediction files for further inspection. These features may provide new insight into vaccine design for infectious diseases such as HIV/AIDS and CoVid 19 and for personalized cancer immunotherapy.

### EXAMPLES

### Example I: Design and Implementation

### Web Application

Written in Python (v3.7) using Django (v2.2.6) Custommune is an online tool that provides an integrative pipeline (FIG. 1) for prediction and filtration of personalized epitopes.

### Sequence Processing

The Biopython package (Chapman and Chang 2000) is used for translating input sequences, then alignment of translated sequences is performed using the python client of Clustal Omega (REST) web service (Sievers et al. 2011). A consensus of the aligned sequences is generated using the Biopython module with a 50% similarity cutoff. The Biopython “ProteinAnalysis” function is used to estimate physicochemical parameters and secondary structure of the consensus sequence, including: molecular weight, gravity, specific count of amino acids, isoelectric point and fractions of secondary structures.

### Epitope Prediction and Filtration Layers

Custommune is connected with RESTful interface (IEDB-API) (Dhanda et al. 2019) to be used as a platform for using NetMHCpan v4.0 (Jurtz et al. 2017) for HLA-I and HLA-II predictions as well as Bepipred v2.0 (Jespersen et al. 2017) for antibody epitopes prediction. Pandas package (McKinney et al. 2010) is then used to structure epitope sorting tables and allow for comparative filtration. The primary filtration is based on IC50 values, a cutoff of 1000 nm is used to prevent loss of potentially false negatives.

The Los Alamos HIV database (http://www.hiv.lanl.gov/content/immunology) was used to create internal HLA class-specific datasets of previously reported immunogenic epitopes against HIV gag. Using pandas (McKinney et al. 2010), high-affinity epitopes are compared to these datasets to highlight epitopes with previously described immunogenicity. Moreover, another filtration layer is designed to report escape variants by comparing each epitope to an internal database collected from various literature sources including: dataset of HLA-associated polymorphisms in the HIV-1 gag gene as reported by (Brumme et al. 2019), as well as the datasets reported by (Christian et al. 2013) and the datasets of CTL/CD8⁺ and T Helper/CD4⁺ epitope variants and escape mutations reported in Los Alamos HIV database (http://www.hiv.lanl.gov/content/immunology/). Additional filtration is obtained by comparing the epitope location within the gag sequence, to gag regions essential for viral assembly and packaging which tend to be structurally and evolutionarily conserved, as reported by (Shytaj and Savarino 2015). To further refine this filtration, Custommune computes the degree of conservation for each epitope by comparing the epitope sequence to the HIV Sequence Compendium database (Foley et al. 2018) which includes 680 alignments of HIV-1/SIVcpz gag protein sequences. The degree of conservation (Cscore) of each epitope is calculated as a fraction represented by the subset of sequences {s} in which the epitope scored a local alignment of more than 80% using Clustal Omega (Sievers et al. 2011) over the total sequences Stotal in the internal database.

$$Cscore = \frac{\{ s \}}{Stotal}$$

The next layer of filtration selects only epitopes that rank high for multiple alleles in case a multiple-allele input was selected by the user for both HLA classes. For further assessment of the impact of predictable mutations, Custommune computes the effect of these mutations (retrieved from the internal gag sequences database) on the binding affinity of epitopes to the patient HLAs. This refined analysis is performed only on the top three epitopes initially predicted by the tool. By computing affinities to the same allele the user can estimate the impact of mutations in this specific segment on affinity to the restricted allele. The degree of deviation of the mutated version is estimated based on SDaffinities, which are calculated as a standard deviation (SD) of the set of IC50 values for the candidate epitope and its mutant versions. The deviation value is therefore considered to negatively reflect the binding stability of this peptide segment to a restricted allele, in respect to a set of predicted mutant versions of the same segment.

### Structural Validation and Epitope Reporting

The python package PeptideBuilder (Tien et al. 2013) is used for generation of 3D models of top epitopes, while the package LightDock (Jiménez-Garcia et al. 2018; Roel-Touris, Bonvin, and Jiménez-Garcia 2020) is implemented to perform epitope-HLA docking based on the Glowworm Swarm Optimization algorithm (GSO) (Krishnanand and Ghose 2009). Solved structures of HLA-Alleles were collected from the pHLA3D database (Menezes Teles E Oliveira et al. 2019) and The Protein Data Bank (PDB) (Berman et al. 2000). Docking scores are included in the final filtration layer for the highest ranking epitope candidates.

### Final Scoring and Annotation

For highly ranking epitope candidates, a scoring function is designed to account for each filtration layer. In this function each continuous parameter (IC50,DFIRE docking score,Cscore and SDaffinities) is represented by a quantitative value, according to the following rules: 1) the IC50 value is rescaled by calculating its reciprocal multiplied by a weighting factor of 10⁴; 2) docking scores are preceded by a negative sign to weight the negative binding energies of the DFIRE scoring function of LightDock; 3) Cscore is considered as a percentile of the Cscore fraction weighted by a factor of 10³; 4) SDaffinities are preceded by a negative sign to weight the positive values of deviation values. Categorical parameters (escapeM,locationscore and DOverlap) are represented by binary values weighted by a factor of 500 for favorable states while non favorable states are given null values.

Overall the formula to calculate the final ranking (S) can be calculated as follows:

S=10000*(IC50)⁻¹−DFIRE+EscapeM*500+CScore*1000+LocationScore*500−SDaffinities+DOverlap*500

The top three epitopes ranked by S score are further analyzed based on their possible overlap with epitope data sets previously associated with: post-ART control, efficacy in vaccine studies and the lack of reported escape mutations. Finally, predicted antibody epitopes estimated by Bepipred 2.0 (Jespersen et al. 2017) are reported if they overlap with the top candidate epitopes ranked by S score. To allow manual inspection of results, sequence processing data and unfiltered prediction results are provided in a separate section of the results page with a downloading link for a text file.

### Example 2. Therapeutic Predictions

### HIV/AIDS

To test Custommune predictions against manual epitope selection we chose an ongoing multi intervention phase II clinical trial enrolling HIV⁺ individuals (NCT02961829). For the trial, autologous dendritic cells were pulsed with a personalized vaccine designed manually from gag sequences isolated from each patient's virus. In the study groups receiving this vaccine (along with other interventions) the patients showed variable responses, including two individuals who displayed significant control of viral load during analytical treatment interruption (Diaz et al. 2019). Using input data from these treatment groups showed that epitopes predicted by Custommune generally displayed some overlap with those administered in the study (FIG. 4A). Of note, when data were stratified based on virologic response, defined as >1 Log₁₀ Δ viral load set point (i.e. the difference between pre- and post-therapy copies of median HIV-1 RNA/mL of plasma measurements), the non-responders were the only patients for which there was no overlapping prediction between Custommune epitopes and those administered in vivo (FIG. 4B). Moreover, patients who were administered vaccine epitopes highly overlapping (>50%) with those predicted by Custommune, were characterized by higher viral load abatement (FIG. 4C). These data suggest that Custommune can predict epitopes with therapeutic potential.

### Cancer

Custommune pipeline could be applied to design peptide vaccines against selected cancer antigens. For this purpose, we validated custommune predictions against specific antigens of interest to cancer immunotherapy. MUC1 is a promising antigen for triple negative breast cancer (TNBC) immunotherapy. Herein, we compared Custommune affinity predictions with in-vivo IFNγ Elispots of CD8-specific MUC1 responses to a pool of MUC1 peptides (Scheikl-Gatard et al., 2017).

IFNγ Elispots of CD8-specific MUC1 responses from immunized HLA-B*27 mice were used for a correlation study. Splenocytes from 2 mice were restimulated with 2 MUC1 peptides of different lengths (11-mer and 15-mer) (FIG. 5A), and CD4-depleted splenocytes from five mice were restimulated with four different MUC1 peptides of different lengths (9-mer, 10-mer, 11-mer and 15-mer) (FIG. 5 B).

Custoscore predictions for affinity of the studied epitopes correlated strongly (R=0.8464; P=0.008048) with the CTL responses observed upon restimulation of splenocytes with two MUC1 peptides (FIG. 5A). However, IC50 values for the same peptides didn't negatively correlate with IFNγ response values (R=−0.475; P=0.234272),In addition, the Custoscores of four peptides used to restimulate CD4-depleted splenocytes correlated strongly (R=0.7985; P=0.005611) with their in-vivo IFNγ responses (FIG. 5 B). IC50 values of the same peptides also had strongly negative correlation coefficient with the IFNγ response values (R=0.798; P=0.005663).

### REFERENCES

Arai, R., H. Ueda, A. Kitayama, N. Kamiya, and T. Nagamune. 2001. “Design of the Linkers Which Effectively Separate Domains of a Bifunctional Fusion Protein.” Protein Engineering 14 (8): 529-32.
Arya, Rimanshee, Amit Das, Vishal Prashar, and Mukesh Kumar. n.d. “Potential Inhibitors Against Papain-like Protease of Novel Coronavirus (COVID-19) from FDA Approved Drugs.” https://doi.org/10.26434/chemrxiv.11860011.v1.
Berman, H. M., T. N. Bhat, P. E. Bourne, Z. Feng, G. Gilliland, H. Weissig, and J. Westbrook. 2000. “The Protein Data Bank and the Challenge of Structural Genomics.” Nature Structural Biology 7 Suppl (November): 957-59.
Bethune, Michael T., and Alok V. Joglekar. 2017. “Personalized T Cell-Mediated Cancer Immunotherapy: Progress and Challenges.” Current Opinion in Biotechnology. https://doi.org/10.1016/j.copbio.2017.03.024.
Burton, Dennis R. 2019. “Advancing an HIV Vaccine; Advancing Vaccinology.” Nature Reviews. Immunology 19 (2): 77-78.
Chapman, Brad, and Jeffrey Chang. 2000. “Biopython.” ACM SIGBIO Newsletter. https://doi.org/10.1145/360262.360268.
Churchill, Melissa J., Steven G. Deeks, David M. Margolis, Robert F. Siliciano, and Ronald Swanstrom. 2016. “HIV Reservoirs: What, Where and How to Target Them.” Nature Reviews. Microbiology 14 (1): 55-60.
Clementz, Mark A., Zhongbin Chen, Bridget S. Banach, Yanhua Wang, Li Sun, Kiira Ratia, Yahira M. Baez-Santos, et al. 2010. “Deubiquitinating and Interferon Antagonism Activities of Coronavirus Papain-like Proteases.” Journal of Virology 84 (9): 4619-29.
Dales, Natalie A., Alexandra E. Gould, James A. Brown, Emily F. Calderwood, Bing Guan, Charles A. Minor, James M. Gavin, et al. 2002. “Substrate-Based Design of the First Class of Angiotensin-Converting Enzyme-Related Carboxypeptidase (ACE2) Inhibitors.” Journal of the American Chemical Society 124 (40): 11852-53.
Dhanda, Sandeep Kumar, Swapnil Mahajan, Sinu Paul, Zhen Yan, Haeuk Kim, Martin Closter Jespersen, Vanessa Jurtz, et al. 2019. “IEDB-AR: Immune Epitope Database-Analysis Resource in 2019.” Nucleic Acids Research 47 (W1): W502-6.
Diaz R S, Giron L B, Galinskas L, Hunter J, Janini M, Shytaj I L, Cauda R, Sucupira M C, Maricato J, Savarino A. Post-therapy viral set-point abatement following combined antiproliferative and immune-boosting interventions: results from a randomised clinical trial. Journal of virus Eradication 2019. Journal of Virus Eradication OP. 8.6
Foley, Brian Thomas, Bette Tina Marie Korber, Thomas Kenneth Leitner, Cristian Apetrei, Beatrice Hahn, Ilene Mizrachi, James Mullins, Andrew Rambaut, and Steven Wolinsky. 2018. “HIV Sequence Compendium 2018.” https://doi.org/10.2172/1458915.
Gao, Jianjun, Zhenxue Tian, and Xu Yang. 2020. “Breakthrough: Chloroquine Phosphate Has Shown Apparent Efficacy in Treatment of COVID-19 Associated Pneumonia in Clinical Studies.” Bioscience Trends, February. https://doi.org/10.5582/bst.2020.01047.
Hajeer, Ali H., Hanan Balkhy, Sameera Johani, Mohammed Z. Yousef, and Yaseen Arabi. 2016. “Association of Human Leukocyte Antigen Class II Alleles with Severe Middle East Respiratory Syndrome-Coronavirus Infection.” Annals of Thoracic Medicine 11 (3): 211-13.
Jespersen, Martin Closter, Bjoern Peters, Morten Nielsen, and Paolo Marcatili. 2017. “BepiPred-2.0: Improving Sequence-Based B-Cell Epitope Prediction Using Conformational Epitopes.” Nucleic Acids Research 45 (W1): W24-29.
Jia, Mingming, Kunxue Hong, Jianping Chen, Yuhua Ruan, Zhe Wang, Bing Su, Guoliang Ren, et al. 2012. “Preferential CTL Targeting of Gag Is Associated with Relative Viral Control in Long-Term Surviving HIV-1 Infected Former Plasma Donors from China.” Cell Research 22 (5): 903-14.
Jiménez-Garcia, Brian, Jorge Roel-Touris, Miguel Romero-Durana, Miguel Vidal, Daniel Jiménez-González, and Juan Fernández-Recio 2018. “LightDock: A New Multi-Scale Approach to Protein-Protein Docking.” Bioinformatics 34 (1): 49-55.
Jurtz, Vanessa, Sinu Paul, Massimo Andreatta, Paolo Marcatili, Bjoern Peters, and Morten Nielsen. 2017. “NetMHCpan-4.0: Improved Peptide-MHC Class I Interaction Predictions Integrating Eluted Ligand and Peptide Binding Affinity Data.” The Journal of Immunology. https://doi.org/10.4049/jimmunol.1700893.
Kiepiela, Photini, Kholiswa Ngumbela, Christina Thobakgale, Dhanwanthie Ramduth, Isobella Honeyborne, Eshia Moodley, Shabashini Reddy, et al. 2007. “CD8+ T-Cell Responses to Different HIV Proteins Have Discordant Associations with Viral Load.” Nature Medicine 13 (1): 46-53.
Krishnanand, K. N., and D. Ghose. 2009. “Glowworm Swarm Optimization for Simultaneous Capture of Multiple Local Optima of Multimodal Functions.” Swarm Intelligence. https://doi.org/10.1007/s11721-008-0021-5.
Lei, Yao, Furong Zhao, Junjun Shao, Yangfan Li, Shifang Li, Huiyun Chang, and Yongguang Zhang. 2019. “Application of Built-in Adjuvants for Epitope-Based Vaccines.” Peer J 6 (January): e6185.
Li, Weidang, Medha Joshi, Smita Singhania, Kyle Ramsey, and Ashlesh Murthy. 2014. “Peptide Vaccine: Progress and Challenges.” Vaccines. https://doi.org/10.3390/vaccines2030515.
McKinney W, others. Data structures for statistical computing in python. In: Proceedings of the 9th Python in Science Conference. 2010.
Menezes Teles E Oliveira, Deylane, Rafael Melo Santos de Serpa Brandão, Luiz Claudio Demes da Mata Sousa, Francisco das Chagas Alves Lima, Semiramis Jamil Hadad do Monte, Mário Sérgio Coelho Marroquim, Antonio Vanildo de Sousa Lima, et al. 2019. “pHLA3D: An Online Database of Predicted Three-Dimensional Structures of HLA Molecules.” Human Immunology 80 (10): 834-41.
Morris, Caroline, Sarah J. Glennie, Hon S. Lam, Holly E. Baum, Dhinushi Kandage, Neil A. Williams, David J. Morgan, Derek N. Woolfson, and Andrew D. Davidson. 2019. “A Modular Vaccine Platform Combining Self-Assembled Peptide Cages and Immunogenic Peptides.” Advanced Functional Materials. https://doi.org/10.1002/adfm.201807357.
Riviére, Yves, Michael B. McCHESNEY, Françoise Porrot, Françoise Tanneau-Salvadori, Philippe Sansonetti, Olga Lopez, Gilles Pialoux, et al. 1995. “Gag-Specific Cytotoxic Responses to HIV Type 1 Are Associated with a Decreased Risk of Progression to AIDS-Related Complex or AIDS.” AIDS Research and Human Retroviruses. https://doi.org/10.1089/aid.1995.11.903.
Roel-Touris, Jorge, Alexandre M. J. J. Bonvin, and Brian Jiménez-Garcia. 2020. “LightDock Goes Information-Driven.” Bioinformatics 36 (3): 950-52.
Savarino, Andrea, Livia Di Trani, Isabella Donatelli, Roberto Cauda, and Antonio Cassone. 2006. “New Insights into the Antiviral Effects of Chloroquine.” The Lancet Infectious Diseases 6 (2): 67-69.
Shytaj, Iart Luca, Gabrielle Nickel, Eric Arts, Nicholas Farrell, Mauro Biffoni, Ranajit Pal, Hye Kyung Chung, et al. 2015. “Two-Year Follow-Up of Macaques Developing Intermittent Control of the Human Immunodeficiency Virus Homolog Simian Immunodeficiency Virus SIVmac251 in the Chronic Phase of Infection.” Journal of Virology 89 (15): 7521-35.
Shytaj, Iart Luca, and Andrea Savarino. 2015. “Cell-Mediated Anti-Gag Immunity in Pharmacologically Induced Functional Cure of Simian AIDS: A ‘Bottleneck Effect’?” Journal of Medical Primatology 44 (5): 227-40.
Sievers, Fabian, Andreas Wilm, David Dineen, Toby J. Gibson, Kevin Karplus, Weizhong Li, Rodrigo Lopez, et al. 2011. “Fast, Scalable Generation of High-Quality Protein Multiple Sequence Alignments Using Clustal Omega.” Molecular Systems Biology 7 (October): 539.
Stephenson, Kathryn E. 2018. “Therapeutic Vaccination for HIV.” Current Opinion in HIV and AIDS. https://doi.org/10.1097/coh.0000000000000491.
Tien, Matthew Z., Dariya K. Sydykova, Austin G. Meyer, and Claus O. Wilke. 2013. “PeptideBuilder: A Simple Python Library to Generate Model Peptides.” Peer J 1 (May): e80.
Velavan, Thirumalaisamy P., and Christian G. Meyer. 2020. “The COVID-19 Epidemic.” Tropical Medicine & International Health. https://doi.org/10.1111/tmi.13383.
Vincent, Martin J., Eric Bergeron, Suzanne Benjannet, Bobbie R. Erickson, Pierre E. Rollin, Thomas G. Ksiazek, Nabil G. Seidah, and Stuart T. Nichol. 2005. “Chloroquine Is a Potent Inhibitor of SARS Coronavirus Infection and Spread.” Virology Journal 2 (August): 69.
Wang, Manli, Ruiyuan Cao, Leike Zhang, Xinglou Yang, Jia Liu, Mingyue Xu, Zhengli Shi, Zhihong Hu, Wu Zhong, and Gengfu Xiao. 2020. “Remdesivir and Chloroquine Effectively Inhibit the Recently Emerged Novel Coronavirus (2019-nCoV) in Vitro.” Cell Research, February. https://doi.org/10.1038/s41422-020-0282-0.
Xiong, P., X. Zeng, M. S. Song, S. W. Jia, M. H. Zhong, L. L. Xiao, W. Lan, et al. 2008. “Lack of Association between HLA-A, -B and -DRB1 Alleles and the Development of SARS: A Cohort of 95 SARS-Recovered Individuals in a Population of Guangdong, Southern China.” International Journal of Immunogenetics. https://doi.org/10.1111/j 0.1744-313x.2007.00741.x.
Yang, Junbao, Eddie James, Michelle Roti, Laurie Huston, John A. Gebe, and William W. Kwok. 2009. “Searching Immunodominant Epitopes prior to Epidemic: HLA Class II-Restricted SARS-CoV Spike Protein Epitopes in Unexposed Individuals.” International Immunology 21 (1): 63-71.
Zhang, Lei, and Yunhui Liu. 2020. “Potential Interventions for Novel Coronavirus in China: A Systematic Review.” Journal of Medical Virology. https://doi.org/10.1002/jmv.25707.
Zhou, Peng, Xing-Lou Yang, Xian-Guang Wang, Ben Hu, Lei Zhang, Wei Zhang, Hao-Rui Si, et al. 2020. “A Pneumonia Outbreak Associated with a New Coronavirus of Probable Bat Origin.” Nature, February. https://doi.org/10.1038/s41586-020-2012-7.
Zhu, Zhongyu, Samitabh Chakraborti, Yuxian He, Anjeanette Roberts, Tim Sheahan, Xiaodong Xiao, Lisa E. Hensley, et al. 2007. “Potent Cross-Reactive Neutralization of SARS Coronavirus Isolates by Human Monoclonal Antibodies.” Proceedings of the National Academy of Sciences of the United States of America 104 (29): 12123-28.
Zuñiga, Rosario, Aldo Lucchetti, Patricia Galvan, Shyla Sanchez, Carmen Sanchez, Ana Hernandez, Hugo Sanchez, et al. 2006. “Relative Dominance of Gag p24-Specific Cytotoxic T Lymphocytes Is Associated with Human Immunodeficiency Virus Control.” Journal of Virology 80 (6): 3122-25.
Scheikl-Gatard T, Tosch C, Lemonnier F, Rooke R. Identification of new MUC1 epitopes using HLA-transgenic animals: implication for immunomonitoring. J Transl Med. 2017; 15(1):154. Published 2017 Jul. 5.

We claim:

1. A personalized vaccine for HIV/AIDS or cancer, wherein peptide affinity is predicted for the candidate vaccine's HLA Class I and/or Class II antigens.

2. The personalized vaccine as in claim 1, comprising an immune epitope, wherein the immune epitope has peptide affinity for HLA predicted using an algorithm excluding protein portions presenting documented immune escape mutations.

3. The personalized vaccine as in claim 2, wherein the algorithm includes predicting epitopes which are least likely to mutate into peptides with decreased affinity for the candidate vaccine's HLA.

4. The personalized vaccine as in claim 3, wherein candidate peptides are scored taking into account both (a) the algorithm's prediction of peptide affinity for HLA excluding protein portions presenting documented immune escape mutations, and (b) the algorithm's prediction of epitopes which are least likely to mutate into peptides with decreased affinity for the candidate vaccine's HLA.

5. The personalized vaccine as in claim 4, wherein the algorithm includes the affinity of the potential escape mutants being calculated by the amplitude of the affinity of the theoretical mutants.

6. A method of providing a set of peptides for use as anti-coronavirus vaccine comprising the steps of:
A) collecting the most prevalent Class II HLAs within a human population/community, and
B) conducting predictions for binding of peptides derived from receptor-binding domain RBD1 and RBD2 regions of SARS Coronavirus-2 (SARS-CoV-2) S-glycoprotein spanning amino acids 397-437 and 455-492 (ref. Wuhan isolate).

7. The method as set forth in claim 6, further comprising forming a vaccine with the peptides, wherein the peptides showing high affinity for the prevalent HLA's within the target population overlap with theoretical or documented neutralizing antibody epitopes.

8. The method as set forth in claim 6, wherein the vector is a viral vector for expression within the human body or endogenous dendritic cells cultivated and thereafter pulsed ex vivo with the very peptides.

9. The method as set forth in claim 6, wherein a suitable vector is virus-like particles (VPLs).

10. The method as set forth in claim 6, administered with a vaccine adjuvant such as alum and/or another similar adjuvant.

11. The method as set forth in claim 6, administered conjugated to gold nanoparticles.

12. The method as set forth in claim 6, wherein said peptides are covalently linked through linker peptide sequences to form a multi-epitope protein.

13. A web tool or software for personalized vaccine design following the algorithm based on: A) input of an individual/population's nucleic acid sequences (HLA I, HLA II, viral sequences of interest); B) nucleic acid translation, sequence alignment, consensus amino acid sequence building; C) epitope predictions and scoring according to claim 1.

14. The personalized vaccine as in claim 1, wherein the vector is a viral vector for expression within the human body or endogenous dendritic cells cultivated and thereafter pulsed ex vivo with the very peptides.

15. The personalized vaccine as in claim 1, wherein a suitable vector is virus-like particles (VPLs).

16. The personalized vaccine as in claim 1, administered with a vaccine adjuvant such as alum and/or another similar adjuvant.

17. The personalized vaccine as in claim 1, administered conjugated to gold nanoparticles.

18. The personalized vaccine as in claim 1, wherein said peptides are covalently linked through linker peptide sequences to form a multi-epitope protein.
